(19) United States
(12) Patent Application Publication
(10) Pub. No.: US 2020/0305690 A1
(43) Pub. Date: Oct. 1, 2020

(54) CLEARING AN ENDOSCOPIC SURGICAL FIELD

(71) Applicant: Massachusetts Institute of Technology, Cambridge, MA (US)

(72) Inventors: Matthew Roy Johnson, Medford, MA (US); Ian Thomas Hill, Wausau, WI (US)

(21) Appl. No.: 16/307,097

(22) PCT Filed: Mar. 29, 2018
(86) PCT No.: PCT/US18/25090

§ 371 Date: Dec. 4, 2018

Related U.S. Application Data
(60) Provisional application No. 62/480,088, filed on Mar. 31, 2017.

(51) Int. Cl. A61B 1/00; A61B 1/018; A61B 1/313; A61B 1/045; A61B 1/005

(52) U.S. Cl. CPC A61B 1/00087; A61B 1/018; A61B 1/3135; A61B 1/0057; A61B 1/045; A61B 1/00137; A61B 1/00006

(57) ABSTRACT

A sheath at the end of an endoscope may be elastically expanded to clear an area within a body cavity during a medical procedure and retracted to within an envelope of the endoscope when not in use. In the expanded state, the sheath may provide sufficient mechanical strength to clear space for a camera or surgical tool, while also having sufficient pliability to mitigate impact on surrounding, sensitive tissue. In an example embodiment, the sheath may be an elastomeric sheath or the like coupled between portions of coaxial shafts of an endoscope, and the coaxial shafts can move relative to one another to deploy or retract the sheath by relatively tensioning and relaxing opposing ends of the sheath. In this manner, the sheath may be selectively expanded and retracted without exposed moving parts or sharp edges.

### CROSS-REFERENCE TO RELATED APPLICATIONS

This application is a national stage entry application of International Patent Application No. PCT/US18/25090 filed on Mar. 29, 2018, which claims priority to U.S. Provisional Application No. 62/480,088, filed Mar. 31, 2017, where the entire contents of each of the foregoing are incorporated herein by reference.

### GOVERNMENT LICENSE RIGHTS

This invention was made with Government support under Contract No. FA8721-05-C-0002 awarded by the U.S. Air Force. The Government has certain rights in the invention.

### TECHNICAL FIELD

The present disclosure generally relates to providing a clearing for a surgical instrument in a surgical field and, more specifically, relates to providing a clearing for an endoscope in relatively compressed anatomy.

### BACKGROUND

In a typical minimally invasive procedure, a surgical tool and a camera may be deployed through an endoscope for use at a treatment site. However, tissue or other obstructions can block a surgeon's view of the treatment site while operating the surgical tool and, in some instances, might impede use of the surgical tool itself. Thus, there remains a need for a tool that clears a treatment site to provide improved visibility and access, particularly in regions of relatively constricted and delicate anatomy such as the spinal column.

### SUMMARY

A sheath at the end of an endoscope may be elastically expanded to clear an area within a body cavity during a medical procedure and retracted to within an envelope of the endoscope when not in use. In the expanded state, the sheath may provide sufficient mechanical strength to clear space for a camera or surgical tool, while also having sufficient pliability to mitigate impact on surrounding, sensitive tissue. In an example embodiment, the sheath may be an elastomeric sheath or the like coupled between portions of coaxial shafts of an endoscope, and the coaxial shafts can move relative to one another to deploy or retract the sheath by relatively tensioning and relaxing opposing ends of the sheath. In this manner, the sheath may be selectively expanded and retracted without exposed moving parts or sharp edges.

In one aspect, an endoscope disclosed herein may include a first elongate shaft having a first distal portion, a second elongate shaft coaxial with the first elongate shaft, the second elongate shaft having a second distal portion extending distally beyond the first distal portion of the first elongate shaft, a camera disposed on the second elongate shaft, and a sheath extending from the first distal portion of the first elongate shaft to the second distal portion of the second elongate shaft, the first distal portion of the first elongate shaft and the second distal portion of the second elongate shaft movable relative to one another to move the sheath between a retracted state and an expanded state, the sheath in the retracted state having a maximum radial dimension that does not substantially exceed a maximum radial dimension of the first distal portion of the first elongate shaft, and the sheath in the expanded state forming a substantially frustoconical surface about the camera.

A relative distance between the first distal portion of the first elongate shaft and the second distal portion of the second elongate shaft may be controllable to control the shape of the substantially frustoconical surface. The camera may have a field of view, and movement of the sheath from the retracted state to the expanded state directs the sheath toward the field of view of the camera. In the absence of an external force exerted on the sheath, the substantially frustoconical surface may be disposed along at least a portion of the field of view of the camera. The substantially frustoconical surface may extend distally beyond the second distal portion of the second elongate shaft. The substantially frustoconical surface may extend distally beyond the camera. The camera may be substantially flush with the second distal portion of the second elongate shaft. The endoscope may further include a tool movable distally beyond the second distal portion of the second elongate shaft, the tool actuatable along a proximal portion of the first elongate shaft, where the substantially frustoconical surface formed by the sheath in the expanded state extends distally beyond the tool. The tool may include one or more of an optical fiber, a drill, a grasper, scissors, a hook, and an expandable paddle. At least a portion of the sheath may fold over itself to form the substantially frustoconical surface as the sheath moves from the retracted state to the expanded state. The sheath may form a seal against entry of fluid into the first distal portion of the first elongate shaft. The sheath in the expanded state may maintain the shape of the substantially frustoconical surface in response to a force greater than about 0.3 g/mm² and less than about 2.0 g/mm². A durometer of the sheath may be less than a respective durometer of one or both of the first elongate shaft and the second elongate shaft. The sheath may have a coefficient of friction less than a respective coefficient of friction of one or both of the first elongate shaft and the second elongate shaft. The sheath may form at least a portion of an outer surface of a distal tip of the endoscope in the retracted state. The second elongate shaft may be shorter than the first elongate shaft. The endoscope may further include at least two pull wires coupled to the second elongate shaft. The at least two pull wires may be actuatable individually to steer the second elongate shaft. The at least two pull wires may be actuatable together to move the sheath between the retracted state and the expanded state.

In another aspect, an endoscope disclosed herein may include a first elongate shaft having a first proximal portion and a first distal portion, a second elongate shaft having a second proximal portion and a second distal portion, the second distal portion of the second elongate shaft extending distally beyond the first distal portion of the first elongate shaft, and a sheath coupled to each of the first distal portion of the first elongate shaft and the second distal portion of the second elongate shaft, the first distal portion of the first elongate shaft and the second distal portion of the second elongate shaft movable relative to one another to expand the sheath relative to the first elongate shaft.

At least a portion of the second elongate shaft may be retractable into the first elongate shaft to radially expand the sheath. The first distal portion of the first elongate shaft and the second distal portion of the second elongate shaft may be axially movable toward one another to expand the sheath. In response to movement of the first distal portion of the first elongate shaft and the second distal portion of the second elongate shaft toward one another, the sheath may be distally movable beyond the second distal portion of the second elongate shaft. In response to movement of the first distal portion of the first elongate shaft and the second distal portion of the second elongate shaft toward one another, the sheath may be expandable to form a circumferential surface about the second distal portion of the second elongate shaft. The circumferential surface may be a substantially frustoconical surface. At least a portion of the sheath may fold over itself to form the circumferential surface. The endoscope may further include a camera disposed along the second distal portion of the second elongate shaft, where the camera has a field of view in a direction distal to the second distal portion of the second elongate shaft, and the sheath is expandable to clear at least a portion of the field of view. The camera may be substantially flush with a distalmost face of the second distal portion of the second elongate shaft. The camera may include a Gaussian optical chain. In response to movement of the first distal portion of the first elongate shaft and the second distal portion of the second elongate shaft toward one another, the sheath may be distally movable beyond the camera. A focal length of the camera may be about 2 mm to about 50 mm. The endoscope may further include a tool movable distally beyond the second distal portion of the second elongate shaft where the expanded surface of the sheath formed by the sheath in the expanded state extends radially and distally beyond the tool. The tool may include one or more of an optical fiber, a drill, a grasper, scissors, a hook, and an expandable paddle. The endoscope may further include a hand-steering mechanism having a stiffness that dominates stiffness of the tool. The tool may have a bending radius of greater than about 10 mm and less than about 15 mm. Between the first distal portion of the first elongate shaft and the second distal portion of the second elongate shaft, the sheath may form a seal against entry of fluid into the first distal portion of the first elongate shaft. The sheath may extend about an entire circumference of the first distal portion of the first elongate shaft and about an entire circumference of the second distal portion of the second elongate shaft. The first distal portion of the first elongate shaft and the second distal portion of the second elongate shaft may be movable relative to one another such that a maximum outer diameter of the sheath is less than or substantially equal to a maximum outer diameter of the first distal portion of the first elongate shaft. The maximum outer diameter of the first distal portion of the first elongate shaft may be about 3 mm or less. The first elongate shaft may define a lumen extending from the first proximal portion to the first distal portion, and at least a portion of the second elongate shaft may be disposed in the lumen. The sheath may be formed of at least one polymer. The sheath may be formed of at least one of a nylon braid or silicone. The sheath may block one or more of ultraviolet energy and infrared energy. At least one of the first elongate shaft and the second elongate shaft may define an irrigation lumen and the sheath may define a plurality of irrigation holes in fluid communication with the irrigation lumen. The first elongate shaft and the second elongate shaft may be substantially coaxial with one another.

In another aspect, a method of medical treatment with an endoscope disclosed herein may include advancing an endoscope to a treatment site of a patient, the endoscope including a first distal portion of a first elongate shaft, a second distal portion of a second elongate shaft, the second distal portion extending distally beyond the first distal portion, and a sheath coupled to each of the first distal portion and the second distal portion; moving the first distal portion relative to the second distal portion to expand the sheath, the radial expansion of the sheath clearing at least a portion of a field of view of a camera disposed along the second distal portion of the second elongate shaft; and based at least in part on an image from the camera, actuating a tool extending through at distal portion of the endoscope at the treatment site.

Advancing the endoscope to the treatment site may include moving the endoscope into epidural space through a sacral hiatus of the patient. The radial expansion of the sheath may clear dura matter from at least a portion of a field of view of the camera. Advancing the endoscope to the treatment site may include moving the endoscope in a retracted state through anatomy of the patient, where the sheath has a maximum radial dimension less than or about equal to a maximum radial dimension of the first distal portion. Moving the first distal portion relative to the second distal portion may include withdrawing the second distal portion proximally toward the first distal portion. The expanded sheath may include a substantially frustoconical surface disposed about the camera. Actuating the tool may include steering the tool within a volume defined by the expanded sheath. The tool may include an optical fiber and actuating the tool may include delivering ablation energy to the treatment site through the optical fiber. Delivering ablation energy to the treatment site through the optical fiber may include directing photochemical energy toward tissue at the treatment site. The sheath may block photochemical energy delivered by the optical fiber and scattered at the treatment site. The method may further include retracting the expanded sheath into a retracted state, repositioning the endoscope at a second treatment site, and repeating the step of moving the first distal portion of the first elongate shaft and the second distal portion of the second elongate shaft relative to one another to expand the sheath.

In another aspect, an endoscope disclosed herein may include a first elongate shaft having a first proximal portion and a first distal portion, a second elongate shaft having a second proximal portion and a second distal portion, the second distal portion of the second elongate shaft substantially aligned with the first distal portion of the first elongate shaft on an end of the endoscope, and a sheath coupled to at least one of the first distal portion of the first elongate shaft and the second distal portion of the second elongate shaft, the first distal portion of the first elongate shaft and the second distal portion of the second elongate shaft movable relative to one another to expand the sheath relative to the first elongate shaft on the end of the endoscope.

In another aspect, an endoscope disclosed herein may include a first elongate shaft having a first proximal portion and a first distal portion, a second elongate shaft having a second proximal portion and a second distal portion, the second distal portion of the second elongate shaft extending distally beyond the first distal portion of the first elongate shaft, and a sheath formed on the first distal portion of the first elongate shaft, the first distal portion of the first elongate shaft and the second distal portion of the second elongate shaft movable relative to one another to expand the sheath on the first distal portion of the first elongate shaft when the second distal portion of the second elongate shaft is disposed within the first elongate shaft.

### BRIEF DESCRIPTION OF THE DRAWINGS

The foregoing and other objects, features and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein.

FIG. 1 is a schematic representation of an endoscopy system during an endoscopic medical procedure on a spine.

FIG. 2A is a perspective view of a distal portion of an endoscope of the endoscopy system of FIG. 1, with the endoscope including a sheath shown in a retracted state.

FIG. 2B is a perspective view of the distal portion of the endoscope of the endoscopy system of FIG. 1, with the sheath shown in an expanded state.

FIG. 2C is a cross-section of a distal portion of the endoscope of the endoscopy system of FIG. 1, taken along the cross-section 2C-2C in FIG. 2A.

FIG. 3 is a flow chart of an exemplary method of operating an endoscope.

FIG. 4 is a cross-section of a distal portion of an endoscope including an irrigation lumen.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are incorporated by reference in their entirety. References to items in the singular should be understood to include items in the plural, and vice versa, unless explicitly stated otherwise or clear from the context. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the embodiments.

In the following description, it is understood that terms such as “first,” “second,” “proximal,” “distal,” and the like, are words of convenience and are not to be construed as limiting terms unless specifically stated to the contrary.

As used herein, unless otherwise indicated or made clear from the context, the term “physician” should be understood to include a surgeon preparing for and/or performing any one or more of the medical procedures described herein and, more broadly, should be understood to include any medical personnel, such as nurses, assisting a surgeon in preparing for or performing any one or more of the medical procedures described herein. Further, as used herein, the term “patient” shall be understood to include any type of mammal, including a human, on which a medical procedure can be performed.

Further, unless otherwise specified or made clear from the context, the terms “surgery” and “surgical” shall be understood to include or be related to any manner and form of invasive medical procedures, whether or not carried out in whole or in part by a surgeon, for the purpose of any one or more of medical diagnosis or medical treatment of a patient. Thus, for example, a “surgical field” shall be understood to include, in the anatomy of a patient, a local region in which an endoscope is used to carry out or assist in a medical procedure. In the context of the present disclosure, minimally invasive surgery or minimally invasive surgical procedures shall be understood to refer to a class of procedures in which a patient's anatomy is accessed using techniques that are less invasive (e.g., requiring smaller incisions) than comparable anatomic access achieved using open surgery. Thus, the term minimally invasive may depend on the context of a particular type of procedure. For example, in certain medical procedures, a minimally invasive procedure may include introduction of an endoscope into the patient via a small incision, such as an incision less than about 20 mm (e.g., less than about 10 mm or less than about 5 mm).

The term “endoscope,” as used herein, shall be generally understood to include any elongate device introducible into, and removable from, a patient as part of a minimally invasive surgical procedure directed toward diagnosis and/or treatment of a condition. Thus, unless otherwise specified or made clear from the context, the term endoscope shall be understood to include any of various different medical probes introducible into anatomy of a patient to provide a physician with direct visualization (e.g., camera-based feedback) or indirect visualization (e.g., feedback based on pressure and/or electrical activity) of an anatomic region of interest. Additionally, or alternatively, endoscopes described herein may include, without limitation, a subset of endoscopes used in the context of abdominal procedures and commonly referred to as laparoscopes.

Endoscopes including clearing tools are described herein in the context of medical procedures related to the spine. It should be appreciated, however, that the devices, systems, and methods described herein shall be understood to be useful in any manner and form of medical diagnosis medical treatment, or combination thereof, associated with confined anatomic regions in which collateral damage along the anatomic region is a significant risk, both in terms of probability and complications. Accordingly, unless another intention is indicated or made clear from the context, the devices, systems, and methods described herein shall be understood to be useful for carrying out any manner and form of endoscopic procedures, which shall be understood to be inclusive of laparoscopic procedures. Further, or instead, it will be understood that the principles described herein may be useful in other contexts, such as in instances in which clearing a view is required or useful for a tool remotely operating in a confined space and, more specifically, in instances in which collateral damage along the confined space is a concern.

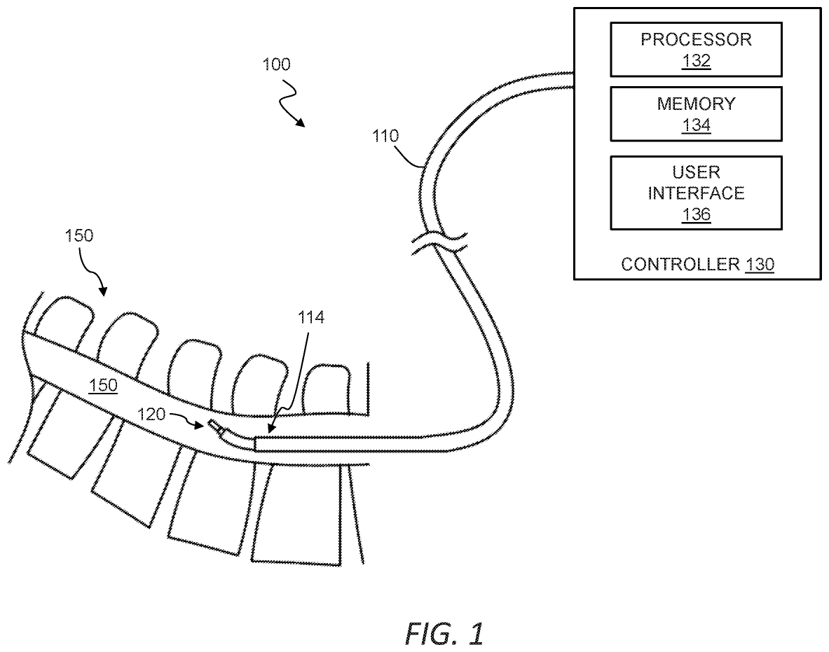

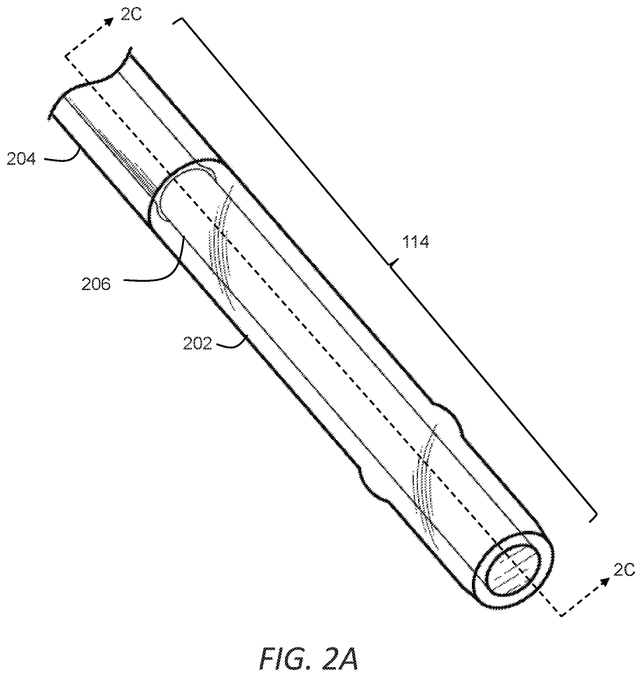

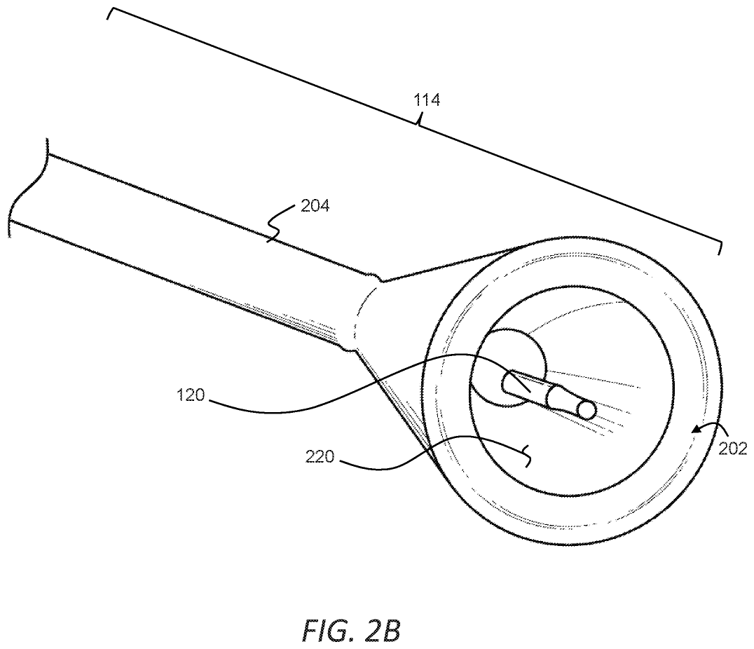

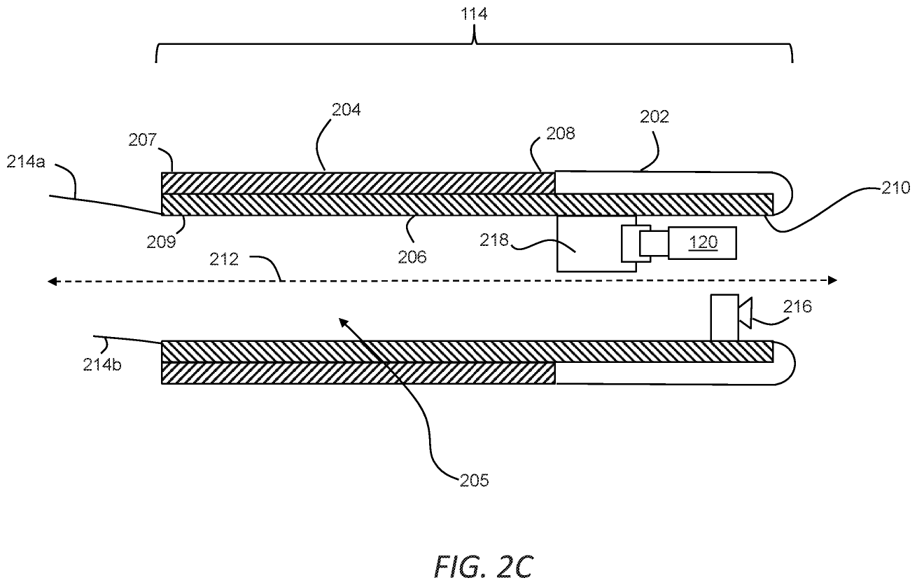

Referring now to FIGS. 1-2C, an endoscopic system 100 may include an endoscope 110, a surgical tool 120, and a controller 130. The endoscope 110 may be electrically coupled to the controller 130 to facilitate visualization of a surgical field as described in greater detail below. The endoscope 110 may be mechanically and/or electrically coupled to the controller 130 for navigation of the endoscope 110 (e.g., along a tortuous path) to a target site of the anatomy for a surgical evaluation or procedure. More specifically, the controller 130 may include a user interface 136 through which a physician guides the endoscope 110 to the treatment site. In certain instances, the physician may operate the user interface 136 to generate electrical signals, mechanical actuation, and combinations thereof to position and otherwise interact with the surgical tool 120 to carry out a medical procedure. Advantageously, the endoscope 110 may include a sheath 202 along a distal end portion 114 of the endoscope 110 and, via the user interface 136, the physician may move the sheath 202 between a retracted state (FIG. 2A) and an expanded state (FIG. 2B). As described in greater detail below, the sheath 202 in the retracted state may have a low profile useful for moving the endoscope to a treatment site as part of a minimally invasive procedure and, in the expanded state, the sheath 202 may move tissue or otherwise clear a surgical field in the vicinity of the distal end portion 114 of the endoscope 110. In the expanded state, the sheath 202 may balance competing considerations of force and collateral tissue damage as a surgical field is cleared in the vicinity of tissue. More specifically, the sheath 202 may be deployable with a force sufficient to clear the surgical field while exhibiting pliability sufficient to yield to tissue in or near the surgical field. Thus, the sheath 202 may clear the surgical field with little or no collateral damage to the tissue, which may be particularly useful in procedures performed in the vicinity of delicate tissue.

The endoscope 110 may be used to perform a medical procedure in a confined anatomic region of a patient. For example, in a surgical procedure performed on the spine 150, an incision may be made in a patient's back (posterior) and, with the sheath 202 in the retracted state, the endoscope 110 may be inserted into a spinal canal 152 of the spine 150, where any of various different medical procedures may be performed. In certain implementations, the endoscope 110 may provide visual access useful for properly positioning the surgical tool 120 and, in some instances, for achieving effective interaction between the surgical tool 120 and the target tissue. As described in greater detail below, such visual access may be facilitated by deploying the sheath 202 at the treatment site to clear material away from the surgical tool 120, thus improving access to the target tissue for the surgical tool 120. Further, or instead, in the expanded state, the sheath 202 may shield non-target tissue from collateral damage associated with actuation of the surgical tool 120. In some medical procedures, such as spinal decompression to treat spinal stenosis, the endoscope 110 may be introduced through the sacral hiatus of the patient and into the spinal epidural space. Continuing with this example, with the endoscope 110 in position at a target site in the spinal epidural space, the sheath 202 may be expanded to clear at least a portion of a surgical field defined between the surgical tool 120 and the target tissue. With the surgical field cleared, the surgical tool 120 (e.g., a laser) may be actuated to deliver ablation energy suitable for removing compressive ligamentum flavum from the affected vertebrae.

The endoscope 110 may include a first elongate shaft 204 and a second elongate shaft 206. The first elongate shaft 204 may be coaxial with the second elongate shaft 206. Further, or instead, the first elongate shaft 204 may define a lumen slidably disposed about an exterior of the second elongate shaft 206 so that the first elongate shaft 204 and the second elongate shaft 206 are movable relative to one another to move the sheath 202 between the retracted position and the expanded position. The first elongate shaft 204 may have a first proximal portion 207 and a first distal portion 208, and the second elongate shaft 206 may have a second proximal portion 209 and a second distal portion 210. The sheath 202 may be coupled to a first distal portion 208 of the first elongate shaft 204 and to a second distal portion 210 of the second elongate shaft 206. In certain instances, the sheath 202 may extend about an entire circumference of the first distal portion 208 of the first elongate shaft 204 and about an entire circumference of the second distal portion 210 of the second elongate shaft 206, which may facilitate, for example, clearing a surgical field substantially independently of orientation of the sheath 202 relative to the surgical field. Further, or instead, with the sheath 202 coupled to the first distal portion 208 of the first elongate shaft 204 and to the second distal portion 210 of the second elongate shaft 206 in this manner, the sheath 202 may form a seal against entry of fluid into the first distal portion 208 of the first elongate shaft 204. Such a seal may be useful, for example, for facilitating cleaning (e.g., sterilizing) the endoscope following a medical procedure. Further, or instead, such a seal may protect surrounding tissue from becoming snagged in the relative movement of the first elongate shaft 204 and the second elongate shaft 206.

In a retracted state, the sheath 202 may be stored in a low-profile orientation relative to the first elongate shaft 204 and the second elongate shaft 206 such that the sheath 202 does not add significantly to the overall size of the distal end portion 114 of the endoscope 110. As an example of such a low-profile orientation, in the retracted state, the sheath 202 may have a maximum outer diameter less than or substantially equal to a maximum outer diameter of the first distal portion 208 of the first elongate shaft 204 (e.g., about 5 mm or less or about 3 mm or less in certain applications, such as spinal applications). As described in greater detail below, movement of the first distal portion 208 of the first elongate shaft 204 and the second distal portion 210 of the second elongate shaft 206 relative to one another may vary the direction and/or nature of forces on the sheath 202 to control the sheath 202 between the retracted state and the expanded state.

The first elongate shaft 204 may be disposed about the second elongate shaft 206, with the first elongate shaft 204 and the second elongate shaft 206 coaxially arranged along a center axis 212 defined by each of the first elongate shaft 204 and the second elongate shaft 206 along at least the distal end portion 114 of the endoscope 110. The second elongate shaft 206 may define at least one lumen 205 through which any of various different components described herein may be moved or controlled as necessary or useful for carrying out any one or more of the techniques described herein. Thus, for example, any one or more of the surgical tools 120 described herein may be movable between the second proximal portion 209 and the second distal portion 210 of the second elongate shaft 206, as part of any one or more of the medical procedures described herein.

The coaxial arrangement of the first elongate shaft 204 and the second elongate shaft 206 to move the sheath 202 may offer significant advantages for carrying out minimally invasive surgical procedures in the vicinity of delicate tissue. For example, the use of the first elongate shaft 204 and the second elongate shaft 206 to actuate movement of the sheath 202 may be implemented without sharp, or otherwise irregular, edges. Thus, it should be appreciated that the coaxial arrangement of the first elongate shaft 204 relative to the second elongate shaft 206 may be useful for robust transmission force to the sheath 202 in a manner that is generally compatible with achieving the clinical outcomes characterized by successful treatment with little or no collateral damage to tissue adjacent to a target area. Additionally, or alternatively, the coaxial arrangement of the first elongate shaft 204 relative to the second elongate shaft 206 may be useful for achieving a spatially efficient arrangement of components. That is, the coaxial arrangement of the first elongate shaft 204 relative to the second elongate shaft 206 may facilitate forming the endoscope 110 with functionality suitable for carrying out a given medical procedure while meeting overall size constraints associated the minimally invasive nature of the medical procedure carried out using the endoscope 110.

In general, the first elongate shaft 204 and the second elongate shaft 206 may have any of various different stiffnesses useful for achieving, in the endoscope 110, an aggregate stiffness suitable for a given medical procedure. As used herein, stiffness refers generally to a degree to which a structural member resists deformation in response to an applied force and may be quantified as durometer on any of various different suitable scales (e.g., Shore A, Shore B, Shore C, or any other scale suitable for characterizing resistance to deformation of the material being characterized). Thus, stiffness should be understood to refer to both rigid and flexible structural members, as the case may be. The shafts should also have suitable compressive and tensile strength to deploy and retract a sheath as contemplated herein. For example, the first elongate shaft 204 and the second elongate shaft 206 may have relatively low stiffness such that the endoscope 110 may, in turn, have flexibility suitable for movement through a tortuous anatomic path. Material composition, wall thickness, overall diameter, and length are examples of parameters that may be varied to achieve respective target stiffnesses of the first elongate shaft 204 and the second elongate shaft 206. Accordingly, by way of non-limiting example, one or both of the first elongate shaft 204 and the second elongate shaft 206 may include polytetrafluoroethylene (PTFE) or another biocompatible material known in the art as suitable for the formation of endoscopic surgical tools and components. As a further or alternative example, the first elongate shaft 204 and the second elongate shaft 206 may have different wall thicknesses, as may be useful for augmenting an aggregate stiffness of the endoscope 110 within an overall size envelope dictated by the nature of the medical procedure.

The first elongate shaft 204 and the second elongate shaft 206 may be movable relative to one another in an axial direction along the center axis 212 to move the sheath 202 between the retracted position (e.g., where the sheath 202 is drawn taught along the exterior of the second elongate shaft 206) and the expanded position (e.g., where the ends of the sheath 202 are brought closer together to elastically deform the sheath 202 into a shape expanding at least in a radial direction). The sheath 202 may have a stiffness less than a respective stiffness of one or both of the first elongate shaft 204 and the second elongate shaft 206. That is, in terms of a quantifiable parameter, the sheath 202 may have a durometer less than a respective durometer of one or both of the first elongate shaft 204 and the second elongate shaft 406 measured on a given durometer scale. Continuing with this example, movement of the first elongate shaft 204 and the second elongate shaft 206 relative to one another results in a change in shape of the sheath 202 while the first elongate shaft 204 and the second elongate shaft 206 remain substantially undeformed by the force imparted to the sheath 202. That is, more generally, the shape of the sheath 202 may be controlled substantially independently of the shape of the first elongate shaft 204 and the second elongate shaft 206 by moving the two shafts relative to one another.

In certain implementations, the first elongate shaft 204 and the second elongate shaft 206 may be moved relative to one another through manual controls operated by a physician to control the shape of the sheath 202. For example, the second distal portion 210 of the second elongate shaft 206 may extend distally beyond the first distal portion 208 of the first elongate shaft 204, and the endoscope 110 may include at least two pull wires 214a, 214b coupled to the second elongate shaft 206. The user interface 136 of the controller 130 may be coupled (e.g., mechanically coupled) to the at least two pull wires 214a, 214b.

In use, the physician may control movement of the second elongate shaft 206 via control inputs (e.g., mechanical inputs) provided to the user interface 136 and delivered to the second elongate shaft 206 via the at least two pull wires 214a, 214b. More specifically, the at least two pull wires 214a, 214b may be pulled individually to steer the second elongate shaft 206, which may impart an overall steering direction to the distal end portion 114 of the endoscope 110. In certain implementations, the at least two pull wires 214a, 214b may be pulled simultaneously to retract the second distal portion 210 of the second elongate shaft 206 into the first distal portion 208 of the first elongate shaft 204. More generally, while pull wires are described herein, any suitable mechanism for applying axial forces to one or both of the first elongate shaft 204 and the second elongate shaft 206 may be used as a mechanism to control deployment and stowage of the sheath 202 as contemplated herein.

As an axial distance along the center axis 212 between the first distal portion 208 of the first elongate shaft 204 and the second distal portion 210 of the second elongate shaft 206 is shortened by retraction actuated by tension on the at least two pull wires 214a, 214b, a compressive force is exerted on the sheath 202. In response to the compressive force, the sheath 202 may expand from the retracted state to the expanded state. From the expanded state, the sheath 202 may be returned to the retracted state through a tensile force produced, for example, by pushing the second distal portion 210 of the second elongate shaft 206 to lengthen the axial distance along the center axis 212 between the first distal portion 208 of the first elongate shaft 204 and the second distal portion 210 of the second elongate shaft 206. While controlled, axial movement along the center axis 212 has been described as a technique for controlling expansion and retraction of the sheath 202, other types of movement may be additionally or alternatively used to control movement of the sheath 202. For example, the first elongate shaft 204 and the second elongate shaft 206 may be rotated relative to one another about the center axis 212 to produce rotational forces on the sheath 202 to furl and unfurl the sheath 202 between the retracted state and the expanded state.

In certain implementations, movement of the first distal portion 208 of the first elongate shaft 204 and the second distal portion 210 of the second elongate shaft 206 relative to one another may be restricted to discrete positions corresponding to discrete positions of the sheath 202. As an example, the discrete positions may correspond to the retracted state and to the expanded state of the sheath 202. Discrete positions may facilitate, for example, manipulation of the sheath 202 by a physician using a simplified user interface associated with the tool control system 140. Further or instead, movement of the first distal portion 208 of the first elongate shaft 204 and the second distal portion 210 of the second elongate shaft 206 relative to one another may control the shape of the sheath 202 to any one or more of various different intermediate positions—which may be discrete or continuous—between the retracted state and the expanded state. Controlling the shape of the sheath 202 to any one or more of various different intermediate positions may be particularly useful, for example, for providing the capability to exert a dynamic range of forces on tissue in the vicinity of the treatment site. Further, or instead, controlling of the shape of the sheath 202 through intermediate positions may advantageously provide a physician with fine control over the amount of force, within the dynamic range, exerted by the sheath 202 on tissue during a medical procedure.

In general, the sheath 202 may be expandable to a shape compatible with a medical procedure performed by the endoscope 110. Thus, for example, the sheath 202 may be expandable to a shape including a radial component greater than a maximum radial dimension of the first distal portion 208 of the first elongate shaft 204 and/or greater than a maximum radial dimension of the second distal portion 210 of the second elongate shaft 206. So dimensioned, the sheath 202 may expand to push tissue in a radial direction to clear a surgical field. Additionally, or alternatively, the sheath 202 may be expandable to a shape including an axial component beyond the second distal portion 210 of the second elongate shaft 206 such that the sheath 202 may expand to push tissue in an axial direction to clear a surgical field.

In some implementations, in response to movement of the first distal portion 208 of the first elongate shaft 204 and the second distal portion 210 of the second elongate shaft 206 relative to one another (e.g., toward one another in an axial direction), the sheath 202 may be expandable to form a circumferential surface 220 about the second distal portion 210 of the second elongate shaft 206. The circumferential surface 220 shall be understood to include any shape formed by folding at least a portion of the sheath 202 over itself. Further, or instead, the circumferential surface 220 may extend radially beyond a maximum diameter of the first distal portion 208 of the first elongate shaft 204 and the second distal portion 210 of the second elongate shaft 206 and may extend distally beyond at least one of the first distal portion 208 of the first elongate shaft 204 and the second distal portion 210 of the second elongate shaft 206. More generally, the circumferential surface 220 formed by folding at least a portion of the sheath 202 over itself may be any of various different shapes defining a spatial envelope within which at least an aspect of a medical procedure (e.g., visualization, diagnosis, treatment, or a combination thereof) may be carried out. In certain implementations, the circumferential surface 220 may be substantially matched to a field of view of a camera used to visualize the surgical field, as described below. In addition, or in the alternative, the circumferential surface 220 may be substantially matched to the surgical tool 120 (e.g., in the axial direction) such that the surgical tool 120 may remain within an envelope defined by the circumferential surface 220, even as the surgical tool 120 is maximally extended under normal operating conditions.

As an example, in the absence of an external force, the circumferential surface 220 may be a substantially frustoconical surface. In this context, the absence of an external force shall be understood to refer to the state of the circumferential surface 220 in the absence of contact with tissue or, more generally, in the absence of force other than any force that may be applied by the first elongate shaft 204 and the second elongate shaft 206. Further, a substantially frustoconical surface shall be understood to include any one or more of various different shapes characterized by a radial dimension increasing (e.g., monotonically increasing) in a distal direction. The increasing radial dimension characteristic of the substantially frustoconical shape may be useful for efficient movement of tissue, which may be particularly advantageous in procedures performed in the vicinity of delicate tissue. That is, the frustoconical shape may clear a relatively small amount of tissue near the second distal portion 210 of the second elongate shaft 206, which is typically not an area of interest (e.g., because it may not be imageable and/or it may not be reachable by the surgical tool 120). Similarly, the frustoconical shape may clear a larger amount of tissue away from the second distal portion 210 of the second elongate shaft 206, which is typically an area amenable to imaging and/or to accessibility by the surgical tool 120.

The sheath 202 may be formed of any one or more of various different biocompatible materials (e.g., one or more polymers, nylon braids, silicone, and combinations thereof) dimensioned and arranged to provide, in the sheath 202, suitable force responses to carry out the techniques described herein. More specifically, suitable force responses of the sheath 202 may include movement between a retracted state and an expanded state in response to relative movement of the first elongate shaft 204 and the second elongate shaft 206, as described above. Further, or instead, in the expanded state, suitable force responses of the sheath 202 may balance the competing design considerations of being sufficiently stiff to clear tissue from a surgical field while being sufficiently flexible to reduce the likelihood of collateral damage to the tissue being cleared. That is, the sheath 202 may be semi-rigid to a degree appropriate for use in a given anatomic region. For instance, the sheath 202 may be formed of one or more materials dimensioned and arranged such that the sheath 202 may maintain the shape of the circumferential surface 220 (e.g., a substantially frustoconical surface) in response to a force greater than about 0.3 g/mm² and less than about 2.0 g/mm².

While the sheath 202 may be generally useful for clearing tissue from a surgical field as the sheath 202 moves from the retracted state to the expanded state, it should be appreciated that the sheath 202 may further, or instead, protect non-target tissue as a treatment is carried out on target tissue. For example, the sheath 202 may be formed with a strength sufficient to resist mechanical degradation (e.g., tearing) resulting from contact that may occur between the surgical tool 120 and the sheath 202 during the course of a medical procedure. Additionally, or alternatively, in instances in which the surgical tool 120 directs energy to target tissue at a treatment site, the sheath 202 may be substantially impermeable to one or more forms of energy directed to the tissue by the surgical tool 120. In this way, the sheath 202 may block energy from the surgical tool 120 from reaching non-target tissue and, thus, may reduce the likelihood of collateral damage to tissue outside of the surgical field. As a more specific example, the sheath 202 may block one or more of ultraviolet energy and infrared energy, which may be particularly useful in implementations in which the surgical tool 120 includes an optical fiber in optical communication with a laser source.

In certain implementations, the sheath 202 may have a coefficient of friction less than a respective coefficient of friction of one or both of the first elongate shaft 204 and the second elongate shaft 206. The lower coefficient of friction of the sheath 202 may, for example, facilitate introduction of the distal end portion 114 of the endoscope 110 into the patient. Further or instead, the lower coefficient of friction of the sheath 202 may facilitate maneuverability of the distal end portion 114 of the endoscope 110 within relatively compressed anatomy. As an example, the lower coefficient of friction of the sheath 202 may be achieved, at least in part, through a coating on the sheath 202.

In general, the sheath 202 may be formed of an elastomeric or other flexible or viscoelastic material that can be deformed from a relaxed shape by applying external forces and returned to the relaxed shape by removing external forces. In this context, a relaxed shape of the sheath 202 shall be understood to be a biased shape to which the sheath 202 returns as force is removed from the sheath 202. In one aspect, the sheath 202 may have a relaxed shape of a substantially frustoconical cone. In this configuration, the first elongate shaft 204 and the second elongate shaft 206 may be moved axially relative to one another to apply axial tension to the sheath 202 to draw the sheath 202 taught into a substantially cylindrical shape against one of the first elongate shaft 204 or the second elongate shaft 206, and the axial tension may be relaxed to permit the sheath 202 to relax elastically into the frustoconical shape for clearing a surgical view. In another aspect, the sheath 202 may have some other relaxed shape, such as a substantially cylindrical shape matching an outer surface of one or both of the first elongate shaft 204 or the second elongate shaft 206, and the first elongate shaft 204 and the second elongate shaft 206 may be moved relative to one another to apply compressive force to respective ends of the sheath 202 (e.g., by drawing the two ends closer to one another) to deform the sheath 202 from the relaxed shape into an expanded, tensioned shape such as a cone or the like. More generally, any combination of relaxed/tensioned states and any corresponding combination of elastomeric or otherwise flexible three-dimensional shapes suitable for expanding to clear a surgical field and collapsing to store in a low-profile orientation relative to one or both of the first elongate shaft 204 and the second elongate shaft 206 may be used as the sheath 202 as contemplated herein.

In some implementations, the endoscope 110 may include a camera 216 disposed along the distal end portion 114 of the endoscope 110. The camera 216 may be in electrical communication with the controller 130. In use, images obtained by the camera 216 may be relayed to the controller 130 and, in some instances, displayed on the user interface 136. In general, the images may provide feedback to the physician regarding one or more of a position of the distal end portion 114 of the endoscope 110 (e.g., relative to an anatomic landmark), positioning the surgical tool 120 relative to a treatment site, or condition of tissue before or after treatment. Thus, for example, based at least in part on the images relayed to the user interface 136, the physician may actuate the at least two pull wires 214a, 214b concurrently to move the sheath 202 to the expanded state. As used herein, the images recorded by the camera 216 should be understood to include still images, video, or a combination thereof. Further, or instead, the camera 216 may form images based on any of various different techniques suitable for a given medical procedure. Thus, for example, the camera 216 may form images based on visible light, infrared radiation, or any other forms of radiation useful for real-time imaging. As a specific example, the camera 216 may include a Gaussian optical chain. Further, or instead, a focal length of the camera 216 may be about 2 mm to about 50 mm, with focal lengths in this range being particularly useful for, example, for diagnosis or treatment of conditions in the spine. It will be understood, however, that other focal lengths are also or instead possible, and the focal length may be a function of the anatomic region in which the endoscope 110 is used.

The camera 216 may be disposed along the second distal portion 210 of the second elongate shaft 206. For example, the camera 216 may be in a fixed position along the second distal portion 210 of the second elongate shaft 206. Such fixed positioning may be useful, for example, for maintaining the camera 216 in a fixed orientation relative to another component of the endoscope 110 (e.g., relative to the sheath 202, the surgical tool 120, or a combination thereof). In some implementations, the camera 216 may be independently movable along the second distal portion 210 of the second elongate shaft 206. As an example, the camera 216 may be movable through the endoscope 110 once the distal end portion 114 of the endoscope is in place at a target anatomic site. More specifically, one or both of the first elongate shaft 204 and the second elongate shaft 206 may define a lumen (e.g., a dedicated lumen) through which the camera 216 may be moved through the endoscope 110 and into visual communication with the target anatomic site.

The camera 216 may have a field of view directed in a distal direction relative to the distal end portion 114 of the endoscope 110. Thus, continuing with this example, as the distal end portion 114 of the endoscope 110 is moved through the anatomy, the field of view of the camera 216 may provide a physician with a view useful for navigation and, in some instances, for identifying a treatment site. Unless otherwise specified, or made clear from the context, the field of view of the camera 216 should be understood to be at least as large as the surgical field at the treatment site. As an example, the surgical tool 120 may have one or more predetermined dimensions of maximum extension beyond the distal end portion 114 of the endoscope 110, and the camera 216 may have a field of view at least large enough to provide visualization of the surgical tool 120 at the one or more predetermined dimensions of maximum extension.

In general, the camera 216 may be positioned relative to the sheath 202 such that expansion of the sheath 202 from the retracted state to the expanded state may clear at least a portion of the field of view of the camera 216. For example, expansion of the sheath 202 may move the sheath 202 toward and/or through the field of view of the camera 216. As a more specific example, expanding the sheath 202 may move at least a portion of the sheath 202 distally beyond the camera 216. Further, or instead, in the absence of external force applied to the sheath 202—that is, in the absence of forces other than force that may be exerted on the sheath by the first elongate shaft 204 and the second elongate shaft 206—the sheath 202 may be disposed along at least a portion of the field of view of the camera 216, which may be useful for maintaining the field of view substantially free of extraneous material. Thus, through the interaction between the sheath 202 and the camera 216, the sheath 202 should be understood as being useful for actively and passively managing visualization of tissue with the camera 216 which, in turn, may facilitate achieving improved diagnosis and/or treatment of the target tissue.

In certain implementations, the camera 216 may be substantially flush with a distal face of the second distal portion 210 of the second elongate shaft 206. In this position, the field of view of the camera 216 may be unobstructed by the second elongate shaft 206. Further, or instead, this position may facilitate access to the camera 216 for cleaning, repair, replacement, or a combination thereof.

In general, the surgical tool 120 may be any of various different tools useful for interacting with tissue for the purpose of diagnosis and/or treatment. Thus, for example, the surgical tool 120 may include any one or more of an optical fiber, a drill, a grasper, scissors, a hook, or an expandable paddle. The surgical tool 120 may be controllable through corresponding mechanical, electrical, and/or computerized inputs (as appropriate for the type of surgical tool 120) at a proximal end of the endoscope 110. For example, the mechanical and/or electrical inputs may be provided by a physician interacting with the user interface 136 of the controller 130. As a more specific example, the surgical tool 120 may include an optical fiber (e.g., a steerable optical fiber) the physician may actuate an appropriate laser source—which may be part of the controller 130—to deliver ablation energy to tissue, as described in U.S. Pat. App. Pub. No. 2015/0148602, the entire contents of which are incorporated herein by reference.

Positioning and movement of the surgical tool 120 relative to the distal end portion 114 of the endoscope 110 may be generally dependent upon the nature of the surgical tool 120 and the nature of the medical procedure to be carried out using the surgical tool 120. Accordingly, in some instances, the surgical tool 120 may be disposed along the second distal portion 210 of the second elongate shaft 206. In this position, the surgical tool 120 may remain substantially stationary as the surgical tool 120 is used in one or more of diagnosis and treatment of target tissue in the field of view of the camera 216. Additionally, or alternatively, the surgical tool 120 may be advanced beyond the second distal portion 210 of the second elongate shaft 206 toward the treatment site, which may include moving the surgical tool 120 along at least a portion of the field of view of the camera 216. In certain implementations, the surgical tool 120 may be disposed in the second distal portion 210 of the elongate shaft 206 as the distal end portion 114 of the endoscope 110 is inserted into the patient and moved toward a target anatomic site. Delivery of the surgical tool 120 to the anatomic site in this way may be useful, for example, for reducing the amount of time associate with a given procedure. Further, or instead, the surgical tool 120 may be moved through the endoscope 110 after the endoscope 110 is positioned at the anatomic site. That is, the endoscope 110 may serve as a conduit for the delivery of a plurality of surgical tools to the anatomic site, which may be particularly useful in instances in which the type of surgical tool required for a given type of procedure is informed by visual feedback provided by the camera 216.

The surgical tool 120 may be used in coordination with one or both of the sheath 202 and the camera 216. For example, at the treatment site, the sheath 202 may be deployed from the retracted state to the expanded state to clear the field of view of the camera 216. With the sheath 202 in the expanded state, the field of view of the camera 216 may be substantially clear such that images obtained by the camera 216 may provide the physician with useful feedback for identifying target tissue, diagnosing the target tissue, positioning the surgical tool 120 relative to the target tissue, and actuating the surgical tool 120 to carry out a medical procedure. The surgical tool 120 may interact with tissue while the sheath 202 is in the expanded state, which may be useful for protecting non-target tissue. In certain instances, however, the surgical tool 120 may interact with tissue while the sheath 202 is in the retracted state. For example, the sheath 202 may be expanded and retracted to compress certain types of tissue (e.g., fat) that may remain compressed for a period of time, during which the surgical tool 120 may be used to interact with tissue. Using the sheath 202 and the surgical tool 120 in such an intermittent manner may, for example, reduce the likelihood that the sheath 202 may interfere with operation of the surgical tool 120.

The endoscope 110, in certain instances, may include one or more hand-steering mechanisms 218 coupled to the surgical tool 120. In general, the one or more hand-steering mechanisms 218 may be in communication (e.g., mechanical communication) with the controller 130 and, in certain implementations, the hand-steering mechanism 218 may be actuated along a proximal portion of the first elongate shaft 204. Through the user interface 136, the physician may provide inputs to control the one or more hand-steering mechanisms 218 to maneuver the surgical tool 120 substantially independently of movement of the distal end portion 114 of the endoscope 110. That is, the one or more hand-steering mechanisms 218 may have a stiffness that dominates stiffness of the surgical tool 120 and, thus, actuation of the one or more hand-steering mechanisms 218 may move the surgical tool 120 independently within the surgical field. In some instances, the surgical tool 120 may have a bending radius of greater than about 10 mm and less than about 15 mm. Bending radius in this range may be particularly useful for defining a surgical field suitably sized for delicate anatomic regions, particularly those in the spine. As an example, the one or more hand-steering mechanisms 218 may include a plurality of nested tubes moveable relative to one another (e.g., in response to input from the physician) to move the surgical tool 120, as described in U.S. Pat. App. Pub. No. 2015/0148602, the entire contents of which are incorporated herein by reference.

While the one or more hand-steering mechanisms 218 have been described as steering the surgical tool 120, it should be appreciated that the one or more hand-steering mechanisms 218 can further or instead steer one or more other components of the endoscope 110. As an example, the one or more hand-steering mechanisms 218 may be coupled to the camera 216 to steer the camera 216 independently of the surgical tool 120. More specifically, the camera 216 may be steered independently of the surgical tool 120 to provide the physician with the ability to change views of the surgical tool 120, as may be useful for visualizing positioning of the tool, actuation of the tool, or a combination thereof. As an additional or alternative example, the one or more hand-steering mechanisms 218 may be coupled to the camera 216 to steer the camera 216 in coordination with the surgical tool 120. Such coordinated movement may be useful, for example, for providing the physician with a simplified user interface based on a single reference coordinate system.

The controller 130 may generally include a computer or any other similar processing circuitry or the like, such as a processor 132, a memory 134, and the user interface 136. In certain instances, user interface 136 may provide a video feed from the camera 216 and, based at least in part on the video feed, the physician may provide one or more mechanical, electrical, or computerized inputs to the user interface 136 to position the endoscope 110, to actuate the sheath 202, and/or to activate the surgical tool 120 (e.g., to actuate a laser source). More generally, the physician may interact with the controller 130 to carry out any one or more of the methods described herein.

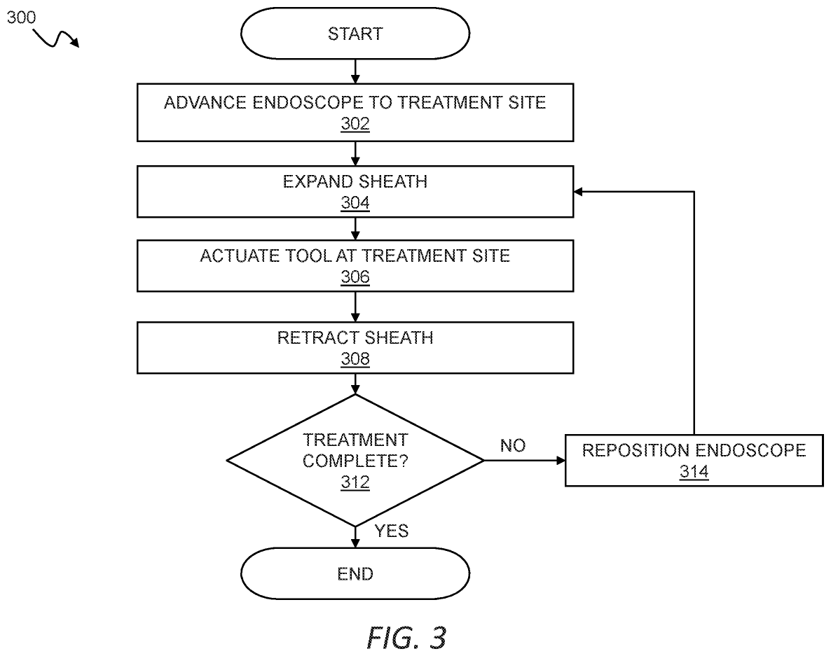

FIG. 3 is a flow chart of an exemplary method 300 for operating an endoscope. Unless otherwise specified or made clear from the context, it should be appreciated that the exemplary method 300 may be carried out using any one or more of the devices, systems, and methods described herein. Thus, for example, the exemplary method 300 may be carried out by the controller 130 (see, e.g., FIG. 1) and, more specifically, can be carried out by a physician providing any manner and form of mechanical inputs, electrical inputs, or computerized inputs to the controller 130 (see, e.g., FIG. 1) to perform a medical procedure using the endoscope 110 (see, e.g., FIG. 1).

As shown in step 302, the exemplary method 300 may include advancing an endoscope to a treatment site of a patient. The endoscope may be any one or more of the various different endoscopes described herein and, more specifically, may include any of various different features of the endoscope 110 shown and described above. Thus, for example, the endoscope may include a first distal portion of a first elongate shaft, and a second distal portion of a second elongate shaft, with the second distal portion extending distally beyond the first distal portion (or otherwise suitably aligned to hold a sheath in a deployed or stowed configuration). The endoscope may, further or instead, include a sheath coupled to each of the first distal portion and the second distal portion. As the endoscope is advanced through the anatomy of the patient, the sheath may be in a retracted state. In the retracted state, the sheath may have a maximum radial dimension less than or about equal to a maximum radial dimension of the first distal portion such that the sheath may have little to no impact on the overall size of the endoscope. In one aspect, the sheath and the elongated shafts may be an independently deployable tool within a body of an endoscope. In another aspect, one or both of the elongate shafts may form an exterior surface of the endoscope, and the sheath may be drawn about an exterior surface of one or both of the elongate shafts when stowed.

As shown in step 304, the exemplary method 300 may include deploying the sheath by expanding the sheath (e.g., by moving the first distal portion of the first elongate shaft relative to the second distal portion of the second elongate shaft). Such relative movement may include, for example, withdrawing the second distal portion of the second elongate shaft proximally toward the first distal portion of the first elongate shaft and, additionally or alternatively, may include moving the first distal portion of the first elongate shaft distally toward the second distal portion of the second elongate shaft. The expansion of the sheath may include expansion in one or more of a radial direction or an axial direction relative to the first elongate shaft according to any one or more of the various different techniques described herein.

In the expanded state, the sheath may form a frustoconical surface or other shape extending in one or more directions away from one or more of the first elongate shaft and the second elongate shaft of the endoscope to clear at least a portion of a field of view of a camera or other tool carried in the second elongate shaft and directed in a direction away from the second elongate shaft. For example, in the context of a spinal procedure, expansion of the sheath may clear dura matter from at least a portion of the field of view of the camera, which may be useful for providing the camera or a surgical tool with access to the treatment site. The expanded sheath can have any of the various different shapes described herein. Thus, for example, the expanded sheath may include a substantially frustoconical surface disposed about the camera, or any other shape that usefully expands relative to another surface of the endoscope to provide a working volume for a surgical procedure.

As shown in step 306, the exemplary method 300 may include actuating a tool at the treatment site. The tool may be any one or more of various different surgical tools useful for diagnosis and/or treatment of tissue at a target site and, more specifically, may include any one or more of the various different surgical tools described herein. Thus, for example, the surgical tool may include an optical fiber in optical communication with a laser source.

The tool may be steered, activated, or otherwise actuated based at least in part on the image from the camera. For example, the physician may use images from the camera as the basis for operating the tool. As a more specific example, the surgical tool may include an optical fiber in optical communication with a laser source, and the physician may use images captured by the camera as the basis for actuating the laser source to deliver energy for a desired therapeutic effect (e.g., to ablate target tissue). In certain implementations, the sheath in the expanded state may usefully block energy delivered by the surgical tool and scattered at the treatment site. That is, returning to the example of the surgical tool including an optical fiber, the sheath in the expanded state may be substantially impermeable to scattered laser energy (e.g., scattered ultraviolet energy, scattered infrared energy, or a combination thereof), thus protecting non-target tissue from unintended exposure to the scattered laser energy.

Actuating the tool may also or instead include steering the tool within a volume defined by the sheath in the expanded state. For example, the tool may be steered substantially independently of a distal end portion of the endoscope and/or substantially independently relative to the camera providing images of the volume. The tool may be steered according to any one or more of mechanical inputs, electrical inputs, or computerized inputs. Thus, for example, steering the tool may include moving the tool based on manual inputs from a physician. Further, or instead, steering the tool may include moving the tool to a set of coordinates through coordinated movement of a plurality of actuators in any automated, semi-automated, or manual control procedure.

As shown in step 308, the exemplary method 300 may include retracting the expanded sheath into a retracted state. In general, the sheath may be retracted according to any one or more of the various different methods described herein. Accordingly, the sheath may be retracted by moving the first shaft and the second shaft relative to one another (e.g., by reversing the movement that resulted in expanding the sheath). In the retracted state, the sheath may have a low profile suitable for movement through anatomy of the patient with a reduced risk of causing collateral damage to tissue.

As shown in step 312, the exemplary method 300 may include repeating one or more of the steps of expanding the sheath, actuating the tool at the treatment site, and retracting the sheath as necessary to complete a treatment. This may include expanding and retracting the sheath at each of a number of locations for a procedure or, where appropriate or helpful, expanding and contracting the sheath multiple times at a single location.

As shown in step 314, the exemplary method 300 may include repositioning the endoscope and repeating the steps of expanding the sheath, actuating the tool at the treatment site, and retracting the sheath. In this way, the endoscope may treat multiple, discrete regions as may be necessary for certain types of medical procedures. Additionally, or alternatively, the endoscope may be repositioned to treat multiple, overlapping regions as may be useful for carrying out certain types of diagnoses and/or treatments.

While certain implementations have been described, other implementations are additionally or alternatively possible.

For example, while the sheath has been described as having a retracted state that does not substantially add to an overall outer dimension of the endoscope, it should be appreciated that other orientations of the retracted state of the sheath are additionally or alternatively possible. For example, a maximum radial dimension of the sheath may be greater than a maximum radial dimension of an outermost elongate shaft of the endoscope. This orientation of the sheath may be useful, for example, in implementations in which the sheath is formed of a soft and/or low-friction material that is particularly well suited for moving through delicate tissue.

As another example, while a coaxial arrangement of shafts has been described as including an inner elongate shaft that is longer than an outer elongate shaft with the sheath in a relaxed state, other relative shaft lengths are additionally or alternatively possible. That is, the inner elongate shaft and the outer elongate shaft may have any of various different lengths relative to one another, with the relative lengths being at least partially dependent upon the relaxed state of the sheath. Thus, in certain implementations, the outer elongate tube and the inner elongate tube may be substantially aligned with one another along the same plane on the end of the endoscope (e.g., a plane that is substantially perpendicular to a center axis defined by one or both of the inner elongate tube and the outer elongate tube). Continuing with this example, the sheath may have a substantially frustoconical shape in the relaxed state, and the sheath may be moved from an expanded state to a retracted state by retracting one of the elongate shafts (e.g., the outer elongate shaft) relative to the other one of the elongate shafts (e.g., the inner elongate shaft). Additionally, or alternatively, the outer elongate shaft may extend distally beyond the inner elongate shaft as the sheath is in the relaxed state, and the outer elongate shaft may be moved proximally to move the sheath from an expanded state to a retracted state.

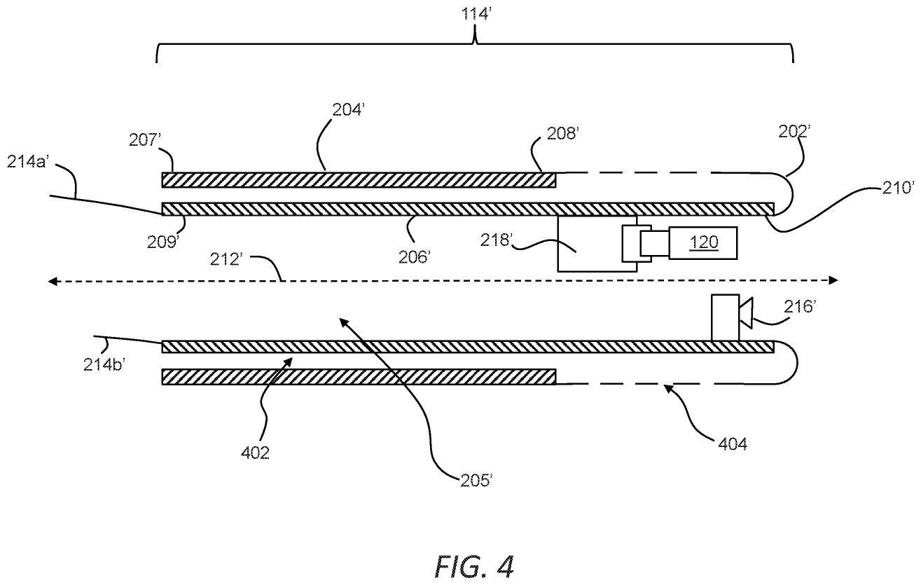

While an inner elongate shaft has been described as defining one or more lumens through which, for example, surgical tools may be moved through the endoscope, other lumen configurations are additionally or alternatively possible. For example, referring now to FIG. 4, a distal end portion 114′ of an endoscope may include a first elongate shaft 204′ and a second elongate shaft 206′ may define a lumen 402 in fluid communication with a sheath 202′ defining a plurality of irrigation orifices 404. Unless otherwise specified or made clear from the context, it should be understood that the distal end portion 114′ may be used interchangeably with the distal end portion 114 (FIGS. 2A-2C) and, thus, may form a portion of the endoscope 110 (FIG. 1). Further, for the sake of efficient description, elements with prime (′) element numbers in FIG. 4 should be understood to be similar to elements with unprimed element numbers in FIGS. 1-2C and are not described separately, except to point out certain differences.

A fluid, such as an irrigation fluid (e.g., saline) or another type of fluid (e.g., a dye), may be introduced into the lumen 402 via a fluid source proximal to the distal end portion 114′ of the endoscope. The fluid may move through the lumen 402, into the sheath 202′, and may exit the sheath 202′ via the plurality of irrigation orifices 404. In certain implementations, movement of the fluid through the plurality of irrigation orifices 404 may at least partially inflate the sheath 202′, which may be useful for expanding the sheath 202′ from a retracted state to an expanded state.

The above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof and may be distributed across devices in a number of ways. At the same time, processing may be distributed across devices such as the various systems described above, or all of the functionality may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y and Z may include any method of directing or controlling any combination of such other individuals or resources to perform steps X, Y and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It should further be appreciated that the methods above are provided by way of example. Absent an explicit indication to the contrary, the disclosed steps may be modified, supplemented, omitted, and/or re-ordered without departing from the scope of this disclosure.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

1. An endoscope comprising:
a first elongate shaft having a first distal portion;
a second elongate shaft coaxial with the first elongate shaft, the second elongate shaft having a second distal portion extending distally beyond the first distal portion of the first elongate shaft;
a camera disposed on the second elongate shaft; and
a sheath extending from the first distal portion of the first elongate shaft to the second distal portion of the second elongate shaft, the first distal portion of the first elongate shaft and the second distal portion of the second elongate shaft movable relative to one another to move the sheath between a retracted state and an expanded state, the sheath in the retracted state having a maximum radial dimension that does not substantially exceed a maximum radial dimension of the first distal portion of the first elongate shaft, and the sheath in the expanded state forming a substantially frustoconical surface about the camera.

2. The endoscope of claim 1, wherein a relative distance between the first distal portion of the first elongate shaft and the second distal portion of the second elongate shaft is controllable to control the shape of the substantially frustoconical surface.

3. The endoscope of claim 1, wherein the camera has a field of view, and movement of the sheath from the retracted state to the expanded state directs the sheath toward the field of view of the camera.

4. The endoscope of claim 3, wherein, in the absence of an external force exerted on the sheath, the substantially frustoconical surface is disposed along at least a portion of the field of view of the camera.

5. The endoscope of claim 1, wherein the substantially frustoconical surface extends distally beyond the second distal portion of the second elongate shaft.

6. The endoscope of claim 1, wherein the substantially frustoconical surface extends distally beyond the camera.

7. The endoscope of claim 1, wherein the camera is substantially flush with the second distal portion of the second elongate shaft.

8. The endoscope of claim 1, further comprising a tool movable distally beyond the second distal portion of the second elongate shaft, the tool actuatable along a proximal portion of the first elongate shaft, wherein the substantially frustoconical surface formed by the sheath in the expanded state extends distally beyond the tool.

9. The endoscope of claim 8, wherein the tool includes one or more of an optical fiber, a drill, a grasper, scissors, a hook, and an expandable paddle.

10. The endoscope of claim 1, wherein at least a portion of the sheath folds over itself to form the substantially frustoconical surface as the sheath moves from the retracted state to the expanded state.

11. The endoscope of claim 1, wherein the sheath forms a seal against entry of fluid into the first distal portion of the first elongate shaft.

12. The endoscope of claim 1, wherein the sheath in the expanded state maintains the shape of the substantially frustoconical surface in response to a force greater than about 0.3 g/mm² and less than about 2.0 g/mm².

13. The endoscope of claim 1, wherein a durometer of the sheath is less than a respective durometer of one or both of the first elongate shaft and the second elongate shaft.

14. The endoscope of claim 1, wherein the sheath has a coefficient of friction less than a respective coefficient of friction of one or both of the first elongate shaft and the second elongate shaft.

15. The endoscope of claim 1, wherein the sheath forms at least a portion of an outer surface of a distal tip of the endoscope in the retracted state.

16. The endoscope of claim 1, wherein the second elongate shaft is shorter than the first elongate shaft.

17. The endoscope of claim 16, further comprising at least two pull wires coupled to the second elongate shaft.

18. The endoscope of claim 17, wherein the at least two pull wires are actuatable individually to steer the second elongate shaft.

19. The endoscope of claim 17, wherein the at least two pull wires are actuatable together to move the sheath between the retracted state and the expanded state.

20. An endoscope comprising:
a first elongate shaft having a first proximal portion and a first distal portion;
a second elongate shaft having a second proximal portion and a second distal portion, the second distal portion of the second elongate shaft extending distally beyond the first distal portion of the first elongate shaft; and
a sheath coupled to each of the first distal portion of the first elongate shaft and the second distal portion of the second elongate shaft, the first distal portion of the first elongate shaft and the second distal portion of the second elongate shaft movable relative to one another to expand the sheath relative to the first elongate shaft.

21-58. (canceled)
